(19) United States
(12) Patent Application Publication
(10) Pub. No.: US 2015/0031629 A1
(43) Pub. Date: Jan. 29, 2015

(54) OPHTHALMIC FORMULATION DERIVED FROM SILK PROTEIN

(71) Applicant: SilkTears, Inc., Plymouth, MN (US)

(72) Inventors: Brian Lawrence, Maple Grove, MN (US); Jon St. Germain, Maple Grove, MN (US)

(73) Assignee: SilkTears, Inc., Plymouth, MN (US)

(21) Appl. No.: 14/513,019

(22) Filed: Oct. 13, 2014

Related U.S. Application Data
(63) Continuation of application No. 14/179,034, filed on Feb. 12, 2014.
(60) Provisional application No. 61/763,882, filed on Feb. 12, 2013.
(60) Provisional application No. 61/824,433, filed on May 17, 2013.

(51) Int. Cl. A61K 38/17; A61K 9/00; A61K 47/36

(52) U.S. Cl. CPC A61K 38/1767; A61K 47/36; A61K 9/0048
USPC 514/20.8

(57) ABSTRACT

An ophthalmic composition is described for the treatment of dry eye syndrome in a human or mammal. The composition comprises an aqueous solution including an effective amount of silk protein. The aqueous solution comprises from about 0.01% by weight to about 30% by weight of the silk protein. In one embodiment, the silk protein may be fibroin. A method of treating an eye having an ocular surface is also described. The method comprises providing an ophthalmic composition comprising an aqueous solution including an effective amount of silk protein, and administering the ophthalmic composition topically to the ocular surface.

### RELATED APPLICATIONS

This application is a continuation of U.S. patent application Ser. No. 14/179,034, filed Feb. 12, 2014, and this application claims priority under 35 U.S.C. §119(e) to U.S. Provisional Patent Application Nos. 61/763,882, filed Feb. 12, 2013, and 61/824,433, filed May 17, 2013, the specifications of which are herein incorporated by reference.

### BACKGROUND

Ophthalmic formulations are described for use as an eye drop for treating a human or other animal and, in particular, artificial tears comprising an aqueous silk protein solution suitable for treating dry eye symptoms.

Keratoconjunctivitis sicca, commonly referred to as “dry eye syndrome”, manifests in the eye as feelings of dryness, burning, or a sandy-gritty sensation. Symptoms of dry eye may also be described as itchy, scratchy, stingy or tired eyes. Other symptoms include pain, redness, a pulling sensation, and pressure behind the eye. Damage to the eye surface resulting from dry eye increases discomfort and sensitivity to bright light. Most sufferers of dry eye experience mild irritation with no long-term effects. However, if the condition is left untreated or becomes severe, dry eye can produce complications that can cause eye damage resulting in impaired vision or possibly loss of vision.

Dry eye is a multi-factorial syndrome that affects the composition of the tear film present on the corneal surface. When the tear film composition is compromised this produces eye irritation if left untreated. Common problems involving tear film composition include increased inflammatory molecule concentration; reduction in the lubricating protein (i.e. mucin) content; reduction of sebaceous oils that prevent water evaporation; and/or reduction in overall tear fluid volume. Any single or combination of these conditions can contribute to dry eye symptoms, and may be caused by a multitude of factors ranging from genetic predisposition, environmental conditions, or injury due to an accident, disease or surgery. As a result, dry eye symptoms are typically treated on multiple levels by providing the patient with various therapies to aid in alleviating symptom causality. These can include prescription drugs, over-the-counter (OTC) eye drops, nutritional supplements, punctual plugs, and various surgical approaches.

The classical model of the tear film anatomy describes three separate and distinct layers consisting of an apical oil layer to limit evaporation and lubricate against the eye-lid, a middle aqueous layer to maintain moisture and thickness, and a basal mucin protein layer to lubricate the cornea's surface and protect the eye's surface from desiccation. More recently with the discovery of a multitude of additional components making up the tear film this classical model has evolved into a more complex and diverse makeup of molecules. These molecules include over a dozen mucin proteins that are responsible for lubricating and protecting the eye. There are also antimicrobial proteins (i.e. lysozyme, lactoferrin); growth factors and suppressors of inflammation (i.e. EGF, IL-1RA); and electrolytes for balancing pH and osmolarity of the tears. As a result, the imbalance in any single or varied number of these molecular entities may result in the development of ocular pathologies, including the symptoms of dry eye. From this perspective dry eye is being recognized as the result of misbalanced tear film content that may arise from a number of potential conditions.

One example of misbalance occurs when mucin protein production is reduced as a result of damage to the cornea's goblet cells as a result of injury, or more commonly, by the aging process. Mucin is a glycoprotein. Mucin provides the basis of the tear film structure and functions to lubricate and protect the ocular surface. Mucin protein is present throughout the aqueous layer and forms an interconnected network of large molecular weight molecules that move across the eye's surface protecting it from both desiccation and the shear stress produced from the eyelid. In addition, it is thought that this network allows for reduced evaporation rate and helps removal of contaminants from the eye's surface. Reduction in mucin content results in greater likelihood of cornea desiccation, infection, and injury.

In addition, increased production of certain molecules present in the tear film that under healthy conditions maintain homeostasis can produce increased inflammation and irritation. This is typically caused by the increased presence of inflammatory cytokines and matrix metalloprotease (MMP) enzymes. Together these molecules can cause increased levels of inflammation, cornea tissue matrix degradation, and ultimately cornea cell death. Since such molecular mechanisms are typically interrelated and dependent on one another the acute symptoms of such molecular imbalance can with time form a chronic state of irritation and worsening vision quality. Imbalances in molecular content typically account for nearly half of all dry eye symptoms.

Abnormal tear composition may also result in the premature destruction of the tears through rapid evaporation of the water content, as the tear gland cannot produce enough fluid to keep up with the dehydration rate. This condition is typically referred to as evaporative dry eye, and may result in tears that have increased salinity and are hypertonic. As a result, the entire conjunctiva and cornea cannot be kept covered with a complete layer of tears during certain activities or in certain environments. It is estimated that over half the dry eye population suffers from some form of evaporative dry eye.

In addition to reduced molecular content, inadequate fluid volume production can cause a reduction in the aqueous tear layer thickness. This thinning of the tear film results in aqueous tear deficiency or lacrimal hypo-secretion, which is typically due to resident inflammation that reduces the channel size that tears flow through. As a result, the lacrimal gland does not produce sufficient tear volumes to keep the entire conjunctiva and cornea covered by a complete fluid layer. Over time such a condition may result in desiccation and damage to the ocular surface. This condition is believed to be prevalent in almost a fifth of all suffering patients.

Conventional treatment of mild and moderate cases of dry eye includes supplemental lubrication. Application of ophthalmic formulations, such as therapeutic eye drops and artificial tears, every few hours can aid in maintaining and strengthening the tear film on the ocular surface and provide temporary relief. Lubricating tear ointments are also used. Tear ointments contain white petrolatum, mineral oil, and similar lubricants, and serve as a lubricant and an emollient.

Ophthalmic formulations for treating dry eye are typically aqueous solutions, which may contain a lubricity or hydration enhancing component, termed demulcents, which include hyaluronic acid (HA), poly-ethylene glycol (PEG), glycerin, hypromellose (HP), and carboxymethyl cellulose (CMC). Certain formulations may contain gel-forming molecules, such as hydroxyl propylene guar (HP-guar) to enhance the efficacy of ophthalmic solutions used on the eye. Other formulations may also be oil-emulsion based chemistries that are utilized for delivering specific drugs to the ocular surface, such as cyclosporine A, for suppressing inflammation occurring in response to tear film hypertonicity. Topical 0.05% cyclosporine A, as a castor oil-based ophthalmic emulsion, is marketed in the United States by Allergan under the trade mark RESTASIS®. The primary purpose of these ophthalmic formulations is to promote increased tear production and thus enhance the overall tear film thickness.

It is believed that due to steric and electrostatic repulsion forces, demulcent molecules have limited interactions with the protein molecules within the tear film. Theoretical conjecture suggests the natural makeup of the tear film, which includes proteins such as mucin, is diluted with such eye drop formulations. As a result, the backbone protein structure that aids in structuring the tear layer may be largely removed. Another significant drawback to eye drops is their lack of residence time on the ocular surface, which also may be due in part to a lack of interaction with both the various molecules that make up the tear film and the ocular surface. The ophthalmic solutions are thus rapidly removed from the ocular surface by blinking and another set of drops must be applied continually to rehydrate the tear film surface.

For the foregoing reasons, there is a need for an ophthalmic formulation for the treatment of dry eye that is not rapidly removed from the surface of the eye by blinking. The new ophthalmic formulation should comprise a structural protein. The structural protein can act as a scaffolding structure to enhance ocular surface residence time and overall tear film stability by interacting with the various molecules in the tear film through numerous charged amino acids.

### SUMMARY

An ophthalmic composition is described for the treatment of dry eye syndrome in a human or mammal. The composition comprises an aqueous solution including an effective amount of silk protein. In one aspect, the aqueous solution comprises from about 0.01% by weight to about 30% by weight of the silk protein, preferably from about 0.1% by weight to about 10% by weight of the silk protein, and more preferably from about 0.5% by weight to about 2% by weight of the silk protein. The silk protein may be fibroin.

In another aspect, the ophthalmic formulation may further comprises as components of the aqueous solution a demulcent agent and a buffering and stabilizing agent. The demulcent agent is selected from hyaluronic acid (HA), hydroxyethyl cellulose, hydroxypropyl methylcellulose, dextran, gelatin, polyols, carboxymethyl cellulose, polyethylene glycol, propylene glycol, hypromellose, glycerin, polysorbate 80, polyvinyl alcohol, and povidone. The demulcent agent is between about 0.01% by weight to about 10% by weight and preferably from about 0.2% by to about 2% by weight. In one aspect, the demulcent agent is HA in an amount of about 0.2% by weight.

In another aspect, the buffering and stabilizing agent is selected from phosphate buffered saline, borate buffered saline, or citrate buffer saline, sodium chloride, calcium chloride, magnesium chloride, potassium chloride, sodium bicarbonate, zinc chloride, hydrochloric acid, sodium hydroxide, and edetate disodium.

In a still further aspect, the ophthalmic formulation further comprises an effective amount of an ophthalmic preservative. The ophthalmic preservative is selected from sodium perborate, polyquad, benzalkonium (BAK) chloride, sodium chlorite, purite, or polexitonium.

In another aspect, the ophthalmic formulation further comprises an effective amount of a vasoconstrictor or an anti-histamine or a combination. The vasoconstrictor and anti-histamine is selected from naphazoline hydrochloride, ephedrine hydrochloride, phenylephrine hydrochloride, tetrahydrozoline hydrochloride, and pheniramine maleate or additional anti-histamine.

In yet another aspect, the ophthalmic formulation further comprises an effective amount of an emollient. The emollient is selected from lanolin, light mineral oil, mineral oil, paraffin, petrolatum, white ointment, white petrolatum, white wax, and yellow wax.

In another aspect, the ophthalmic formulation further comprises an effective amount of an inactive ingredient to enhance material properties. The inactive ingredient is selected from hydroxypropyl guar, xantham gum, and trehalose or additional sugar molecules and derivatives.

A method of treating an eye having an ocular surface is also described. The method comprises providing an ophthalmic composition comprising an aqueous solution including an effective amount of silk protein, and administering the ophthalmic composition topically to the ocular surface.

### BRIEF DESCRIPTION OF THE DRAWINGS

For a more complete understanding of the present invention, reference should now be had to the accompanying drawings and description below. In the drawings:

FIG. 1A-1D are schematic representations of a tear model.

FIG. 2 is a bar graph showing the degree of spreading of ophthalmic formulations.

FIG. 3 is a bar graph showing the contact angle of ophthalmic formulations.

FIG. 4 is a bar graph showing the tear film break-up time of ophthalmic formulations.

### DESCRIPTION

An ophthalmic formulation comprises a blend or mixture of an aqueous silk protein solution for treating the symptoms of keratoconjunctivitis sicca, or dry eye. The silk protein in an aqueous solution phase provides a formulation suitable for topical application to the eye of a human or animal suffering from dry eye for relieving the symptoms thereof. Further, a method is provided for treating dry eye, the method comprising providing an ophthalmic formulation comprising a blend or mixture of an aqueous silk protein solution, and administering the silk protein solution topically to the ocular surface or immediate vicinity of an eye of a patient. The blend or mixture of the aqueous silk protein solution may optionally include a therapeutic molecule or other drug.

A silk protein, fibroin, is derived from the Bombyx mori silkworm cocoon. Fibroin comprises a heavy chain that is up to 400,000 Da in molecular weight. The fibroin protein chains possess hydrophilic N and C terminal domains, and alternating blocks of hydrophobic/hydrophilic amino acid sequences allowing for a mixture of steric and electrostatic interactions with surrounding molecules in solution. At low concentration dilutions (1% or less) the fibroin protein molecule is known to take on an extended protein chain form and not immediately aggregate in solution. In addition, the fibroin protein is highly miscible with hydrating molecules like HA, PEG, glycerin, and CMC, and has been found to be highly biocompatible and integrates or degrades naturally within the body through enzymatic action.

Fibroin can be solubilized in water through a standard set of chemical processing regimes known in the art. Fibroin can be concentrated to over 30% by weight concentration in water. In one method, B. mori silk cocoons (Institute of Sericulture, Tsukuba, Japan) are cut into fourths and boiled for 45 minutes in 0.3 weight % Na₂CO₃ (Sigma-Aldrich) to extract the glue-like sericin proteins from the structural fibroin proteins. The fibroin extract is then rinsed three times in deionized water, dissolved in a four times volume to extracted fibroin fiber dry weight of 9.5M or greater lithium bromide (LiBr) solution heated to 60° C., and set covered within a 60° C. oven for 4 hours. The solution is then dialyzed to remove the LiBr salts using dialysis membranes with a molecular weight cutoff (MWCO) of 3,500 Da or 10,000 Da in water for 48 hours with 6 water changes at 1, 4, 8, 12, 12 and 12 hour subsequent time points. To remove mass particulates the dialyzed solution can then be either centrifuged twice at 10,000 g for 20 munites, and/or filtered through a glass fiber depth filter with pore sizes ranging from 5 μm and above. The supernatant can then be collected and stored at 4° C. The final concentration of aqueous silk solution is typically in the range of 3-9 weight % as determined by gravimetric analysis, and final concentration will depend on specific dialysis time, water volume, and membrane MWCO.

The aqueous fibroin solution may be used in ophthalmic formulations for treating diseases and conditions of the eye. In one embodiment, an ophthalmic formulation comprises an effective amount of an aqueous fibroin solution. In particular, the mixtures of this embodiment of the ophthalmic formulation comprise from about 0.01% to about 30% silk protein by weight and about 70% to about 99.99% water by weight.

In another embodiment, an ophthalmic formulation comprises an aqueous fibroin solution in an aqueous mixture including a lubricating demulcent agent, inactive ingredients to enhance material properties, and buffering and stabilizing agents. The concentration of the silk protein within the ophthalmic formulation is preferably between about 0.01% to about 30% by weight, more preferably between about 0.1% to about 10% by weight, and most preferably between about 0.5% to about 2% by weight. Suitable demulcent agents include, but are not limited to, HA, CMC, PEG, PG, or any additional active ingredients listed on the FDA's OTC monograph guidelines listed at 21 CFR Part 349—Drug Products for Over-the-Counter Human Use, the contents of which are hereby incorporated herein in their entirety. The demulcent agent may be added to the formulation to enhance tear film hydration. The demulcent agents may be optionally added at concentrations preferably between about 0.01% to about 10% by weight, more preferably between about 0.1% to about 5% by weight, and most preferably between about 0.2% to about 2% by weight, or as specified in the range indicated for each entity in 21 CFR Part 349. Suitable buffering and stabilizing agents include, but are not limited to, phosphate buffered saline (PBS), borate buffered saline (BBS), citrate buffer saline (CBS), calcium chloride, magnesium chloride, potassium chloride, sodium bicarbonate, zinc chloride, hydrochloric acid, sodium hydroxide, edetate disodium, or any additional inactive ingredients listed on the FDA's OTC monograph guidelines listed at 21 CFR Part 349—Drug Products for Over-the-Counter Human Use, the contents of which are hereby incorporated herein in their entirety. Optionally, the formulation may include solely or in any combination additional active ingredients such as naphazoline hydrochloride, pheniramine maleate, and additional active ingredients as specified in 21 CFR Part 349. In addition, inactive ingredients may be added to the formulation to enhance material properties and wetting capability. Suitable inactive ingredients may include hydroxypropyl guar, xantham gum, and trehalose or additional sugar molecules and derivatives. Optionally, the ophthalmic formulation may include a preservative, such as sodium perborate, polyquad, benzalkonium chloride, sodium chlorite, purite, polexitonium, or any additional preservative as specified in 21 CFR Part 349. Optionally, the ophthalmic formulation may include a vasoconstrictor or anti-histamine, such as naphazoline hydrochloride, ephedrine hydrochloride, phenylephrine hydrochloride, tetrahydrozoline hydrochloride, and pheniramine maleate or additional anti-histamine, or any additional ingredient specified in 21 CFR Part 349. Optionally, the ophthalmic formulation may include an emollient, such as lanolin, light mineral oil, mineral oil, paraffin, petrolatum, white ointment, white petrolatum, white wax, yellow wax, or any additional ingredient specified in 21 CFR Part 349.

The ophthalmic formulation can be delivered to the eye in the form of an eye drop. In use, the silk fibroin protein acts as an enhanced wetting and structuring agent to better stabilize the tear film upon the ocular surface through hydrostatic, electrostatic, or hydrogen bonding interactions with the tear film molecular components throughout the tear film volume. The ophthalmic formulation serves to effectively coat the eye's surface and prolong the residence time of the drop upon the eye's surface. As a result, the ophthalmic formulation including the aqueous silk protein solution will act to coat the eye and stabilize the tear film, thereby providing a more robust barrier against irritating stimulus to the eye's surface giving relief from dry eye symptoms and improving the quality of vision.

In addition, the silk fibroin ingredient may also impart inherent biomaterial properties upon the eye's surface, such as anti-inflammation and enhanced wound healing through non-specified biological interaction. These non-specific interactions aid in reducing the symptoms of dry eye by reducing inflammation and promoting wound healing rate. By reducing inflammation and promoting wound healing rate the eye drop user will experience enhanced reduction in dry eye symptoms over the time of eye drop use.

### Example I

Silk fibroin protein solution was produced by first cutting silkworm cocoons (Tajima Shoji Co., Ltd., JP) into halves in order to remove the remaining pupae body inside. The cocoon halves were then boiled in 0.3 weight % NaCO₃ solution for 60 minutes in 600 mL of water per gram of cocoon to extract the fibroin protein from the contaminating sericin protein. The silk was then washed four times in similar volumes of deionized water for 20 minutes each. The cocoons were continually agitated throughout both the extraction and washing processes to ensure adequate sericin removal and rinsing. The silk fibers were then dried for 1.5 hours with 60° C. convective air, and then dissolved into a four times volume of around 9.5M LiBr solution (FMC Lithium, Inc., NC) to dry fiber weight. The dissolved silk fibroin and LiBr solution was covered tightly and placed into a 60° C. oven for a 4 hour incubation period. After the incubation period about 5 mL of solution were placed per cm SnakeSkin Dialysis tubing (Thermo-Scientific, Inc., IL) that had a 3,500 MWCO and 35 mm inner diameter measurement. The silk solution was then dialyzed against a 222× volume ratio of deionized water. The water was exchanged in 1, 4, 8, 12, 12, and 12-hour intervals, respectively. The silk protein solution was then removed from the dialysis tubing and centrifuged twice at 10,000 g forces for 20 minutes each at 4° C. Additionally, the silk fibroin solution was also depth filtered using filter paper to remove any remaining gross contaminants. The final concentration of the silk protein solution was determined to be around 5 weight % based on gravimetric analysis using an analytical balance (Mettler-Toledo, Ohio).

The eye drop formulation containing silk protein solution was prepared in the following way. A PBS solution was prepared by mixing PBS salts (Sigma-Aldrich, Inc., MO) in deionized water at a concentration that would provide for a 0.01 M phosphate buffer, 0.0027 M potassium chloride and 0.137 M sodium chloride, pH 7.4, at 25° C. when diluted with the 5 weight % silk fibroin solution to provide a 1 weight % final silk fibroin concentration. The salts were mixed until in solution and then filtered through 0.5 μm glass fiber filter (Advantec, JP). Before the 5 weight % silk fibroin was added, HA (Lifecore, Inc., MN) was dissolved into the PBS solution to create a 0.2 weight % HA concentration in solution. The HA/PBS solution was placed into a 60° C. oven to expedite dissolution of the HA, which took approximately 1 hour. Next, the appropriate volume of 5 weight % silk fibroin solution was added to the HA/PBS solution to provide a 1 weight % silk fibroin and 0.2 weight % HA concentrations. The solution was then pre-filtered with a 0.5 μm glass fiber filter (Advantec, JP) and then sterile filtered into an appropriate eye drop bottle using a PES membrane filter (Millipore, Inc., MA)

The ophthalmic formulation was assessed for lubricity by touch, which indicated the drop was both lubricious and produced a viscous solution. The silk solution was then tested in a human eye. The eye drop formulation was applied to each eye in 25 volunteers to test for comfort. A total of 4 volunteers used the formulation for multiple days, and experienced consistent relieving effects. All volunteers indicated that the formulation felt both comfortable and relieving when compared to leading brand artificial tear products. The ophthalmic formulation proved to be non-irritating and provided a soothing coating effect for up to 12 hours per application. It was shown that clarity of vision was unaffected by subjective survey of the volunteers. At the 1 weight % concentration, silk protein was found to not adhere to the eyelashes, cause blurred vision, or provide discomfort. It was determined that at concentrations at 2 weight % and above appeared to cause some blurred vision upon immediate use, and tended to become stuck in the eye lashes after use.

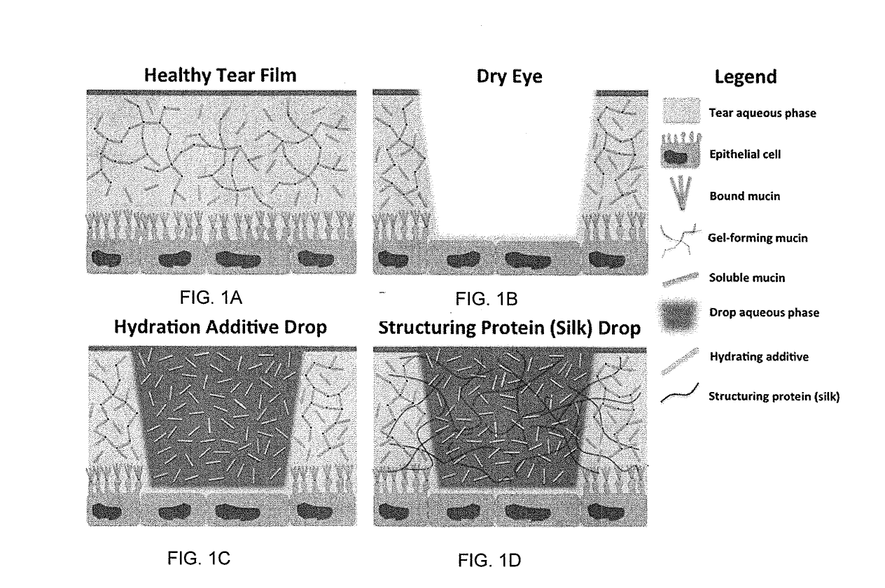

Research has shown that the tear film does not consist of distinct layers, but instead has mucin distributed throughout the aqueous layer over the ocular surface. This is schematically shown in FIG. 1A depicting a healthy tear film. The tear film is significantly reduced during dry eye symptoms, leading to the presence of reduced tear volume over areas of the ocular surface as schematically shown in FIG. 1B. The non-wetted portions on the ocular surface lead to irritation and pain for the individual experiencing the dry eye symptoms.

Conventional ophthalmic formulations function use hydrating eye drops containing demulcent molecules, such as HA, CMC, PEG, glycerin, or PG to promote water retention on the eye's surface. These formulations function optimally with the classical distinct layering model of the tear film. Specifically, the ophthalmic formulations promote the enhancement of the aqueous layer in order to maintain ocular surface hydration and improve lubricity. However, the interactions with the varying protein components of tear film may be limited due to either charge and/or steric repulsion. This is schematically shown in FIG. 1C depicting a tear film containing a standard artificial tear formulation.

The ophthalmic formulations described herein function optimally with the modern understanding of the tear film makeup, where the aqueous region is a more complex mixture of various chemical components distributed throughout the entire tear film volume. Due to the fact that silk fibroin is a protein it can act more interactively with the various components of the tear film due to the varying amount of hydrophobic and hydrophilic amino acids comprising it's structure. This is shown schematically in FIG. 1D showing a tear film with both hydrating and structuring silk protein molecules combined. It is expected that the silk fibroin protein will interact not only with the aqueous components of the tear film, but with the corneal surface as well.

### Example II

A silk fibroin-based formulation consisting by percent weight of 1% silk fibroin protein, 0.2% HA, and PBS buffer was compared against phosphate buffered saline (PBS) solution, Systane® artificial tears formulation by Alcon, Inc., and Blink® artificial tears formulation by AMO, Inc. Various samples (n=3) were dropped onto a Parafilm wax surface to characterize wetting characteristics in which the area of spreading was measured using ImageJ software (NIH, Bethesda, Md.). Various samples (n=4) were dropped onto a Parafilm surface and imaged using a goniometer setup to capture the contact angle of each drop upon the Parafilm wax surfaces. Contact angle was measured using the DropSnake application (EPFL, Lausanne, CH) in ImageJ software. Tear film break up (TFBU) time for each formulation was assessed on wild-type mice (n=3) using standard fluorescein dye assessment to indicate when tear film evaporation has taken place.

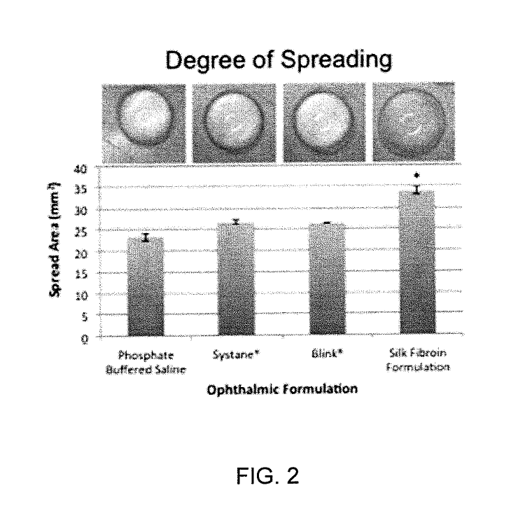

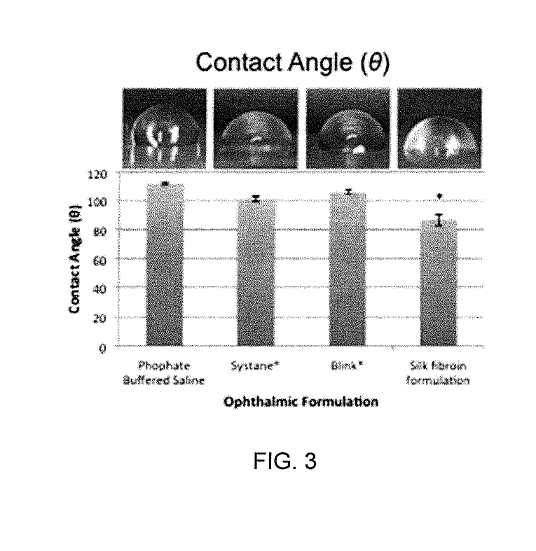

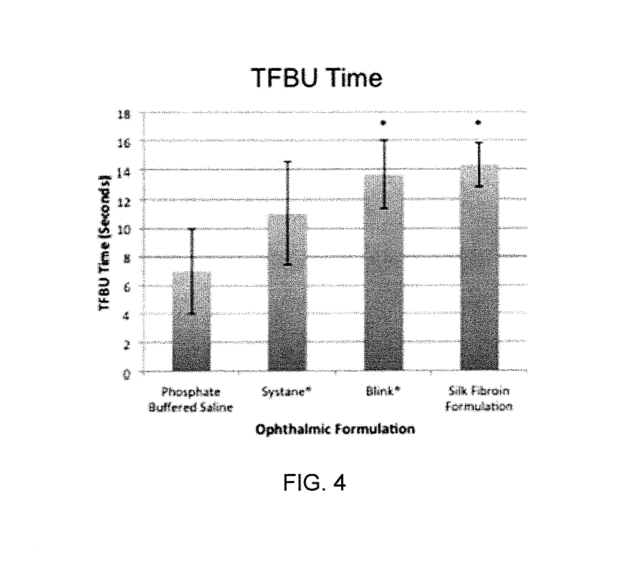

Referring to FIG. 2, the degree of material spreading was significantly higher for the silk protein ophthalmic formulation when compared to the other solutions (n=3, error bars=SD, and * indicates p<0.05 when compared to all other groups). This indicates that the silk protein imparts a coating ability that the other formulations lack. As shown in FIG. 3, the contact angle data demonstrates that silk protein ophthalmic formulations have significantly lower surface energy, which adds further evidence to the fibroin protein's ability to help the solution spread on a hydrophobic surface like the corneal epithelium (n=4, error bars=SD, and * indicates p<0.05 when compared to all other groups). FIG. 4 shows the results of assessing corneal residence time of the silk protein solution upon the cornea surface (n=3, error bars=SD, and * indicates p<0.05 when compared to PBS). Residence time studies indicated that silk protein solution promoted a significant increase (p<0.05) in tear film break up time (TFBU) when compared to PBS controls, had a greater average TFBU than the artificial tear Systane®, and had a comparable TFBU when compared to Blink® artificial tears, which also contains hyaluronic acid. It is has been shown previously that artificial tear products, such as Systane®, have material residence times extending to 2 hours post-application. This data infers that ophthalmic formulations with a silk protein additive may impart greater residence times upon the ocular surface.

These results indicate that a silk fibroin additive in artificial tears helps promote material properties for improved performance of the ophthalmic formulation. Specifically, fibroin protein appears to greatly contribute to ocular surface coating and tear film stabilization. It can be inferred from these results that the fibroin molecules may act as a structuring protein agent to aid the ophthalmic formulation's ability to rewet the ocular surface as schematically represented in FIGS. 1A-1D. While not wishing to be bound by theory, it is believed the ophthalmic formulation comprising silk fibroin interacts with gel forming mucins and aqueous components of the tear film to provide a scaffolding to better stabilize the tear film. The silk fibroin provides a chemical component that has not been a part of prior ophthalmic formulations. That is a large soluble protein, which may be combined with demulcent additives for a more complete supplement for tear film structure and enhance stability.

Ophthalmic formulations comprising an aqueous silk protein solution have many advantages, including inherent coating abilities superior to the leading ophthalmic formulations. Formulations incorporating this additive spread easily over the surface of the eye to provide a protective coating to the ocular surface. It is believed that this ability to spread on an aqueous surface enables the ophthalmic formulation to form a thin layer on the ocular surface, thereby prolonging residence time on the eye. The inherent biocompatibility of the formulation also ensures that an unwarranted immune or inflammatory reaction will not occur with application to the ocular surface. Further, the formulation is biodegraded by enzymes that naturally occur in the tear film and also present within the body, which ensures the formulation is broken down into its amino acid components.

Although the ophthalmic formulation has been described in considerable detail with respect to only a few exemplary embodiments thereof, it should be understood by those skilled in the art that I do not intend to limit the ophthalmic formulation to the embodiments since various modifications, omissions and additions may be made to the disclosed embodiments without materially departing from the novel teachings and advantages of the ophthalmic formulation, particularly in light of the foregoing teachings. Accordingly, I intend to cover all such modifications, omission, additions and equivalents as may be included within the spirit and scope of the ophthalmic formulation as defined by the following claims.

Although the ophthalmic formulation has been described in considerable detail with respect to only a few exemplary embodiments thereof, it should be understood by those skilled in the art that I do not intend to limit the ophthalmic formulation to the embodiments since various modifications, omissions and additions may be made to the disclosed embodiments without materially departing from the novel teachings and advantages of the ophthalmic formulation, particularly in light of the foregoing teachings. Accordingly, I intend to cover all such modifications, omission, additions and equivalents as may be included within the spirit and scope of the ophthalmic formulation as defined by the following claims.

What is claimed is:

1. A method to wet the ocular surface of an eye comprising administering an effective amount of an aqueous ophthalmic composition to the ocular surface of the eye, the composition comprising:
a buffering agent, a demulcent, and a hydrolyzed fibroin protein;
wherein the demulcent comprises about 0.01% to about 10% of the ophthalmic composition by weight, and the hydrolyzed fibroin protein comprises about 0.01% to about 10% of the ophthalmic composition by weight;
wherein the demulcent is hydroxyethyl cellulose, hydroxypropyl methylcellulose, dextran, gelatin, carboxymethyl cellulose, polyethylene glycol, propylene glycol, glycerin, polysorbate 80, polyvinyl alcohol, povidone, or hyaluronic acid;
wherein the ophthalmic composition increases the effectiveness of the spreading of the ophthalmic composition over the ocular surface compared to a corresponding composition that lacks the hydrolyzed fibroin protein; and the ophthalmic composition has a reduced surface tension with respect to the surface of the eye, compared to a corresponding composition that lacks the hydrolyzed fibroin protein.

2. The method of claim 1 wherein the hydrolyzed fibroin protein has been obtained by removal of sericin from silk protein and the resulting fibroin protein has been hydrolyzed in the presence of lithium bromide.

3. The method of claim 1 wherein the buffering agent comprises buffered saline, a chloride salt, or a combination thereof.

4. The method of claim 3 wherein the buffered saline is phosphate buffered saline, borate buffered saline, citrate buffered saline, or a combination thereof.

5. The method of claim 3 wherein the chloride salt is sodium chloride, calcium chloride, magnesium chloride, potassium chloride, zinc chloride, or a combination thereof.

6. The method of claim 1 wherein the aqueous ophthalmic composition comprises one or more additional buffers, one or more additional chloride salts, or a combination thereof.

7. The method of claim 1 wherein the aqueous ophthalmic composition comprises one or more of sodium bicarbonate, hydrochloric acid, sodium hydroxide, and edetate disodium.

8. The method of claim 1 wherein the demulcent comprises about 0.2% to about 2% of the ophthalmic composition by weight.

9. The method of claim 1 wherein the demulcent is hyaluronic acid in an amount of about 0.2% by weight.

10. The method of claim 1 wherein the hydrolyzed fibroin protein comprises about 0.1% to about 10% of the ophthalmic composition by weight.

11. The method of claim 1 wherein the hydrolyzed fibroin protein comprises about 0.5% to about 2% of the ophthalmic composition by weight.

12. The method of claim 1 wherein the aqueous ophthalmic composition comprises an effective amount of an ophthalmic preservative.

13. The method of claim 12 wherein the ophthalmic preservative is selected from sodium perborate, sodium peroxide, phosphoric acid, polyquad, benzalkonium chloride, sodium chlorite, purite, or polexitonium.

14. The method of claim 1 wherein the aqueous ophthalmic composition comprises an effective amount of a vasoconstrictor, an anti-histamine, or a combination thereof.

15. The method of claim 14 wherein the vasoconstrictor or antihistamine is selected from naphazoline hydrochloride, ephedrine hydrochloride, phenylephrine hydrochloride, tetrahydrozoline hydrochloride, or pheniramine maleate.

16. The method of claim 1 wherein the aqueous ophthalmic composition comprises an effective amount of an emollient.

17. The method of claim 16 wherein the emollient is selected from lanolin, light mineral oil, mineral oil, paraffin, petrolatum, white ointment, white petrolatum, white wax, and yellow wax.

18. The method of claim 1 wherein the aqueous ophthalmic composition comprises an inactive ingredient selected from hydroxypropyl guar, xantham gum, trehalose, and sugar molecules and derivatives.

19. A method to wet the ocular surface of an eye comprising administering an effective amount of an aqueous ophthalmic composition to the ocular surface of the eye, the composition comprising:
phosphate buffered saline, borate buffered saline, sodium chloride, magnesium chloride, a demulcent, and a hydrolyzed fibroin protein;
wherein the demulcent comprises about 0.2% to about 2% of the ophthalmic composition by weight, and the hydrolyzed fibroin protein comprises about 0.1% to about 10% of the ophthalmic composition by weight;
wherein the demulcent is hydroxyethyl cellulose, hydroxypropyl methylcellulose, dextran, gelatin, carboxymethyl cellulose, polyethylene glycol, propylene glycol, glycerin, polysorbate 80, polyvinyl alcohol, povidone, or hyaluronic acid;
wherein the ophthalmic composition increases the effectiveness of the spreading of the ophthalmic composition over the ocular surface compared to a corresponding composition that lacks the hydrolyzed fibroin protein; and the ophthalmic composition has a reduced surface tension with respect to the surface of the eye, compared to a corresponding composition that lacks the hydrolyzed fibroin protein; and
wherein the hydrolyzed fibroin protein has been obtained by removal of sericin from silk protein and the hydrolyzed fibroin protein has been hydrolyzed in the presence of lithium bromide.

20. A method to improve the spreading of an ophthalmic composition across the surface of the eye comprising preparing an ophthalmic composition that comprises a demulcent and a hydrolyzed fibroin protein, and administering the ophthalmic composition to the eye.

21. The method of claim 20 wherein the ophthalmic composition comprises:
the demulcent, the hydrolyzed fibroin protein, phosphate buffered saline, borate buffered saline, sodium chloride, magnesium chloride;
wherein the demulcent comprises about 0.2% to about 2% of the ophthalmic composition by weight, and the hydrolyzed fibroin protein comprises about 0.1% to about 10% of the ophthalmic composition by weight;
wherein the demulcent is hydroxyethyl cellulose, hydroxypropyl methylcellulose, dextran, gelatin, carboxymethyl cellulose, polyethylene glycol, propylene glycol, glycerin, polysorbate 80, polyvinyl alcohol, povidone, or hyaluronic acid;
wherein the ophthalmic composition increases the area that the ophthalmic composition covers compared to a corresponding composition that lacks the hydrolyzed fibroin protein; the ophthalmic composition has a reduced surface tension with respect to the surface of the eye, compared to a corresponding composition that lacks the hydrolyzed fibroin protein; and the ophthalmic composition has a longer tear film break-up time compared to a corresponding composition that lacks the hydrolyzed fibroin protein; and
wherein the hydrolyzed fibroin protein has been obtained by removal of sericin from silk protein and the hydrolyzed fibroin protein has been hydrolyzed in the presence of lithium bromide.
